(19) United States
(12) Patent Application Publication
(10) Pub. No.: US 2024/0398591 A1
(43) Pub. Date: Dec. 5, 2024

(54) Implant For Treating Vascular Malformations

(71) Applicant: phenox GmbH, Bochum (DE)

(72) Inventors: Hans HENKES, Stuttgart (DE); Hermann MONSTADT, Bochum (DE); Volker TRÖSKEN, Witten (DE); Ralf HANNES, Dortmund (DE)

(73) Assignee: phenox GmbH, Bochum (DE)

(21) Appl. No.: 18/697,169

(22) PCT Filed: Oct. 4, 2022
(86) PCT No.: PCT/EP2022/077581

§ 371 Date: Mar. 29, 2024

(30) Foreign Application Priority Data
Oct. 1, 2021 (DE) 10 2021 125 571.6

(51) Int. Cl. A61F 2/852; A61F 2/86

(52) U.S. Cl. CPC A61F 2/852; A61F 2/86; A61F 2220/0041; A61F 2220/005; A61F 2220/0058; A61F 2220/0075; A61F 2230/006; A61F 2250/0048

(57) ABSTRACT

The invention relates to a combination implant comprising a flow diverter (1, 2) and at least one stent (3, 4), wherein the flow diverter (1, 2) and the stent (3, 4) form a functional unit (5, 6) and the radial force of the stent (3, 4) is greater than the radial force of the flow diverter (1, 2).

The invention relates to a combination implant having a flow diverter and at least one stent for the treatment of vascular malformations and in particular for the treatment of cerebral aneurysms as well as an implant system for the treatment of cerebral bifurcation aneurysms and a method for its placement.

Vascular malformations can lead to considerable health impairments or even death in a patient. This applies to aneurysms as a common form of such malformations, in particular when they occur in the cerebral area. As a rule, attempts are made to close such malformations with implants. Such implants are usually placed endovascularly with the help of catheters.

Aneurysms that have formed in highly tortuous vessels or a vascular fork, a so-called bifurcation, pose a particular challenge for treatment. Wide-necked aneurysms, for example, also limit the choice of preferred treatment methods. In the case of wide-necked aneurysms, for example, there is a risk that coils inserted to close the aneurysm will be completely or partially flushed out of the aneurysm, leading to vascular occlusion.

In recent years, the use of flow diverters has proven successful in the treatment of such problematic aneurysms in many cases.

Flow diverters are braided, close-meshed, tubular or hose-shaped implants. They initially resemble stents, but unlike stents, they are not intended to revascularize a vessel but to cover the aneurysm neck in order to minimize the blood flow on it and into the aneurysm. Accordingly, unlike a stent, a flow diverter only has a low radial force, which essentially only needs to be suitable for pressing the flow diverter against the vessel wall, and a much narrower mesh that ensures a redirection of the blood flow.

Due to good treatment results, flow diverters are now used for different types of aneurysms.

Due to the positive treatment results achieved to date with flow diverters, it would be accordingly desirable to be able to use them in other indications what was not possible to date due to product-specific disadvantages.

Disadvantages are in particular the low radial forces of known flow diverters, which may lead among others to undesirable deformations, in particular to disadvantageous expansions or constrictions. Corresponding expansions and constrictions may, for example, as a result lead to undesirable obstructions of the blood flow in the vessel.

The use of flow diverters in bifurcations is also not yet possible or not possible with satisfactory results.

One of the difficulties of using flow diverters in bifurcations, for example for the treatment of bifurcation aneurysms, is that the blood flow into the aneurysm is reduced, but the blood flow into one of the draining vessels is not reduced at the same time. However, when placing a flow diverter, which must be done in the inflowing and one of the outflowing vessels in order to bridge the aneurysm, the other outflowing vessel is inevitably also covered by the flow diverter. The flow diverter therefore guides the blood flow around both the aneurysm and the other outflowing vessel.

As a solution, a second flow diverter could be placed from the inflowing vessel into the other outflowing vessel. Due to the low radial forces and the tendency of the flow diverter to dilate or constrict, such an arrangement usually leads to the dominance of one of the two flow diverters over time. The dominant flow diverter will direct a large part of the blood flow into the outflowing vessel it occupies. The other outflowing vessel and the structures dependent on it are no longer adequately supplied with blood by the suppressed flow diverter.

The task of the invention is therefore to provide an implant that does not have the aforementioned disadvantages of known flow diverters.

Furthermore, it is the task of the invention to provide an implant system that enables the use of flow diverters in the treatment of bifurcation aneurysms without the known disadvantages.

This task is solved by a combination implant with the features of claim 1 and an implant system with the features of claim 8. Furthermore, in claims 9 and 10, methods for placing an implant system according to claim 8 are proposed. Advantageous embodiments are each the subject of the dependent claims. It should be noted that the features listed individually in the claims can also be combined with each other in any technologically useful manner and thus demonstrate further embodiments of the invention.

A combination implant according to the invention comprises a flow diverter and at least one stent, wherein the flow diverter and the stent form a functional unit and the radial force of the stent is greater than the radial force of the flow diverter.

The term “functional unit” should be understood to mean that the stent and flow diverter are operatively connected to each other. In particular, the expansion diameters, lengths and radial forces of the stent and flow diverter are coordinated as described in more detail below.

Preferably, the radial force of the stent exceeds the radial force of the flow diverter to such an extent that the radial force of the flow diverter is negligible in relation to the resulting radial force of the combination implant.

The stent may be self-expandable or balloon-expandable. Preferred stent materials for self-expandable stents are nitinol, cobalt-chromium alloys and other metals.

Laser-cut stents may be manufactured as open-cell and closed-cell variants. Closed-cell variants can generally be repositioned more easily or allow the product to be completely replaced during the intervention. Open-cell variants, on the other hand, adapt better to the course of the vascular anatomy.

Radiopaque markers may be provided at different positions of the stent, allowing the user to determine the position and deployment of the stent in the blood vessel.

The stents are designed in such a way that no strut parts protrude into the inner lumen of the stent in the partially expanded state and can thus impair the flow.

Numerous stent incision patterns are conceivable, such as one strut branching into two struts when viewed from proximal to distal, or two struts converging and either merging into one strut or branching again into two struts. Many other stent designs are conceivable for the stents.

Thus, the structure of a stent according to the invention essentially corresponds to known cerebral stents.

The flow diverter essentially corresponds to known flow diverters and is woven from known materials.

Preferably, the flow diverters are braided from 32 to 128 wires, with a braid of 48 to 64 wires being preferred.

As wires for the flow diverter are those made of nitinol, cobalt-chromium alloys and other metals are preferred, that allow self-expansion of the implant. Wire materials that are available as DFT material (Drawn Filled Tubing) with an X-ray visible core (e.g. made of platinum) are also preferred.

Optional, alternatively or additionally, radiopaque markers may be provided on the flow diverter to allow the user to reliably determine the position and deployment of the flow diverter in the blood vessel.

The flow diverter may optionally have a coating, hydrophilic polymer coatings with antithrombogenic or endothelium-promoting properties and/or radiopaque coatings, for example on a gold basis, are preferred here.

The flow diverter may be provided with a membrane to improve flow detour and/or to improve sealing. The membrane covers the entire flow diverter or at least a substantial part of it. The membrane may be provided on the inside and/or outside of the flow diverter. In particular, the membrane may be made of biocompatible materials, such as those known from medical technology. In a preferred embodiment, the membrane is produced by electrospinning.

The combination of a flow diverter and a stent in the combination implant has the advantage that the stent supplements the radial force that the flow diverter lacks and creates an additional structure.

Both embodiments of the combination implant in which the stent surrounds the flow diverter, i.e. the flow diverter is provided within the lumen of the stent, and embodiments in which the flow diverter surrounds the stent, i.e. the stent is provided within the lumen of the flow diverter, are conceivable.

With such a functional arrangement of flow diverter and stent according to the invention, it is clear to the skilled person that the respective (nominal) expansion diameters of flow diverter and stent must also be matched to each other in such a way that the arrangements described above are in an effective connection with each other according to the invention.

The flow diverter and the stent of a combination implant may be provided unconnected and connected. If the flow diverter and the stent are unconnected, this is referred to below as an unconnected combination implant; if the flow diverter and the stent are connected, this is referred to below as a connected combination implant.

Accordingly, an unconnected combination implant is to be understood as a combination implant whose individual elements, namely the flow diverter and stent, are physically unconnected outside the patient's body and are usually initially placed separately from each other during the intervention, i.e. sequentially. Only once inside the patient's body do the individual elements of unconnected combination implants form a functional unit, even without specific connecting elements.

Unconnected combination implants are conceivable, comprising-possibly also complementary-connecting elements on the flow diverter and/or stent, which form a connection after placement in the patient's body.

According to the invention, a connected combination implant is to be understood as such combination implants whose individual elements, namely the flow diverter and stent, are already physically connected outside the patient's body and are placed as a unit during the intervention.

The problem with connected combination implants is that the flow diverter and the stent behave differently during expansion. In particular, the reductions in length during expansion are different, which may inevitably lead to unacceptable tension between the flow diverter and the stent during placement in the vessel. Accordingly, it is not possible to continuously connect the flow diverter and stent over their entire common length.

This problem is solved according to the invention as follows.

In a preferred embodiment of a connected combination implant, the flow diverter and the stent are each connected to each other in the proximal region only. The stent protrudes slightly beyond the flow diverter in the proximal region. This is advantageous in order to prevent a fish mouth effect, i.e. a proximal narrowing of the flow diverter. The stent may optionally also protrude beyond the flow diverter in the distal area.

It is important in this embodiment that the stent, if it is intended for use in a bifurcation, covers at least the section of the combination implant over which the two combination implants will touch during implantation in a bifurcation in order to prevent unwanted expansion of one of the flow diverters. This is usually the area of the proximal start of the combination implant right into the outflowing vessel.

Preferably, the flow diverter is provided outside the stent, i.e. the stent is located in the inner lumen of the flow diverter. However, embodiments in which the stent is provided outside the flow diverter are also conceivable. In the second case, it is advantageous to provide the proximal connection between the stent and the flow diverter in such a way that no fish-mouth effect can occur within the combination implant. The connection between the stent and the flow diverter is then preferably located at the outermost proximal end of the flow diverter.

Variants of this first embodiment are conceivable in which the flow diverter and the stent are interwoven proximally, i.e. the connection consists of a type of entanglement. In addition, adhesive connections, welded connections, knotted, riveted, soldered or other connections are also conceivable for all connections.

In a further embodiment of the invention, an implant system according to the invention comprises at least two combination implants according to the invention, wherein the radial forces of the two combination implants of the implant system are of equal size with the same deployment diameter of the combination implants and thereby result in equally large deployment diameters at least at the proximal ends of the combination implants of the implant system.

This ensures that no combination implant is dominated by the other and that the diameter is narrowed. The dominance of one of the combination implants and the corresponding constriction of the other combination implant lead to an uneven supply to the corresponding vessels. In particular, this may lead to an undersupply or even occlusion of the vessel supplied by the constricted combination implant.

The implant system according to the invention thus comprises at least two combination implants according to the invention for use in the treatment of aneurysms, wherein the radial force A of the first combination implant and the radial force B of the second combination implant are at least proximally equal with the same deployment diameter.

Both connected and unconnected combination implants are suitable for the implant system according to the invention, whereby an implant system preferably comprises only connected or only unconnected combination implants. In individual cases, however, an implant system that comprises a combination of connected and unconnected combination implants as required can also be advantageous.

A method of placing an implant system with two unconnected combination implants, in which the stents are provided within the flow diverters, in a bifurcation comprises the following steps:
(A) Placement of one microcatheter each from proximal through the inflowing vessel in one outflowing vessel each;
(B) Sequential or parallel implantation of the flow diverter or the flow diverters, wherein the proximal end of the respective flow diverter is located in the inflowing vessel and the distal end of the respective flow diverter is located in the respective outflowing vessel.

The microcatheters are each placed again for subsequent implantation of the stents in the respective vessels according to step (A).
(C) Sequential or parallel implantation of the stent or the stents in each case within the corresponding flow diverter, with the proximal end of the respective stent lying proximal to the proximal end of the respective flow diverter. The distal ends of the stents are each located in the outflowing vessels.

The stent protrudes proximally over the flow diverter. This prevents proximal contraction of the flow diverter and a corresponding obstruction of the blood flow. Distally, the stent extends into the respective outflowing vessel and may optionally also protrude distally beyond the flow diverter.

In a further procedure for placing an alternative implant system with two unconnected combination implants, in which the flow diverters are provided inside the stents, the stents are implanted first. After implantation, the flow diverters line the stents from the inside. Here too, the stents protrude proximally over the flow diverters and the stents also extend distally into the outflowing vessels.

The further procedure for placing the alternative implant system with two unconnected combination implants in a bifurcation therefore comprises the following steps:
(AA) Placement of one microcatheter each from proximal through the inflowing vessel in one outflowing vessel each;
(BB) Sequential or parallel implantation of the stent or the stents, whereby the proximal end of the respective stent is located in the inflowing vessel and the distal end of the respective stent is located in the respective outflowing vessel.

The microcatheters are each placed again for subsequent implantation of the flow diverters in the respective vessels according to step (AA).
(CC) Sequential or parallel implantation of the flow diverter or the flow diverters in each case within the corresponding stent, with the proximal end of the respective flow diverter lying proximal to the proximal end of the respective stent and the distal end of the respective flow diverter (1, 2) lying in the respective outflowing vessel (AGA, AGB).

Accordingly, a method of placing an implant system with two connected combination implants for the treatment of a bifurcation aneurysm comprises the following steps:
(AAA) Placement of one microcatheter each (MKA, MKB) from proximal through the inflowing vessel (ZG) in one outflowing vessel (AGA, AGB) each;
(BBB) Sequential or parallel implantation of the connected combination implants, whereby the proximal end of the respective combination implant is located in the inflowing vessel and the distal end of the respective combination implant is located in the respective outflowing vessel.

The combination implants according to the invention, implant systems and methods for their placement are in particular suitable for cerebral bifurcation aneurysms, but may also be used or applied in other parts of the body. The combination implants according to the invention are also not only suitable for the treatment of bifurcation aneurysms, even if this has been dealt with in particular.

The combination implant according to the invention has the advantage over the prior art that the flow diverters combined with a stent as a combination implant have an increased radial force in the desired areas. At the same time, the stents provide the flow diverters in the combination implants with an additional structure or a structural sheath. The stents thus prevent unwanted bulging or indentation of the flow diverters in the corresponding areas.

The invention and the technical environment are explained in more detail below with reference to the figures. It should be noted that the figures show a particularly preferred embodiment of the invention. However, the invention is not limited to the embodiment shown. In particular, the invention includes any combination of the technical features listed in the claims or described in the description as being relevant to the invention, insofar as this is technically feasible.

It shows:

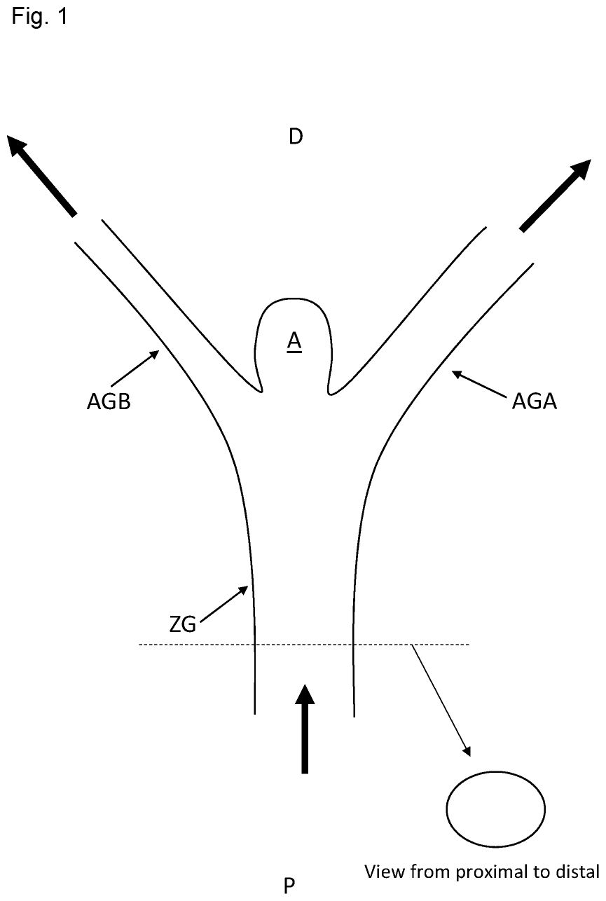

FIG. 1 a schematic representation of a bifurcation aneurysm with the inflowing and outflowing vessels;

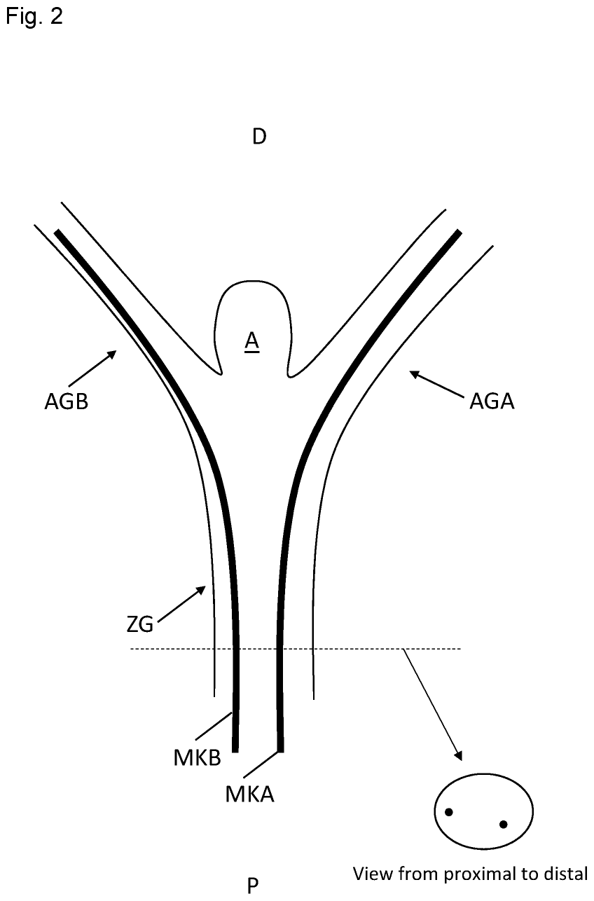

FIG. 2 the bifurcation aneurysm shown in FIG. 1 with the microcatheters in place;

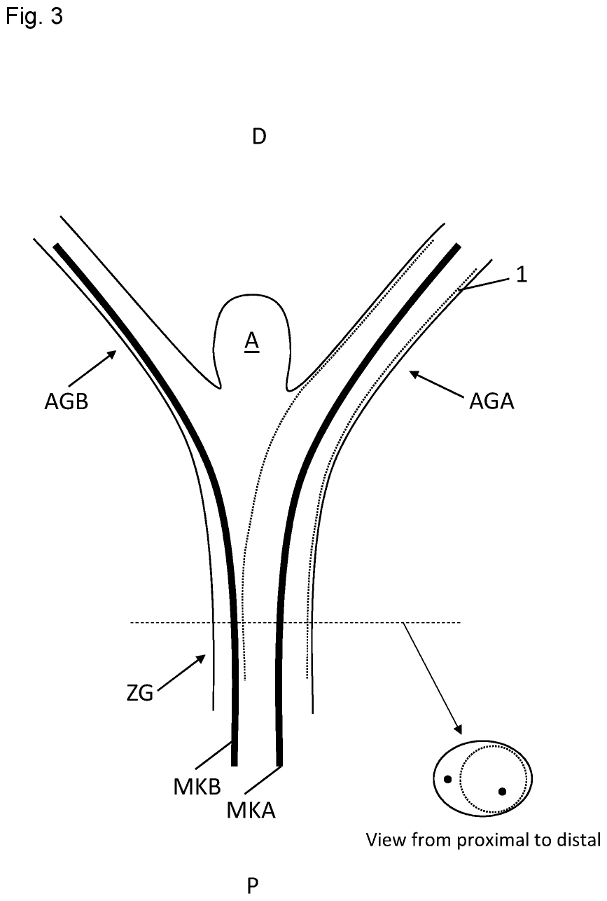

FIG. 3 the bifurcation aneurysm with the microcatheters placed as shown in FIG. 2 and with the first flow diverter placed;

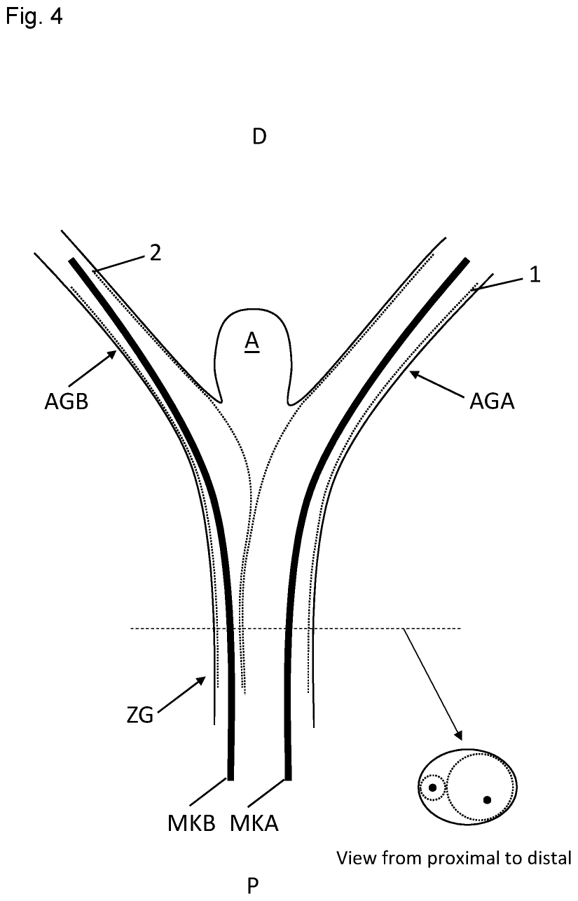

FIG. 4 the bifurcation aneurysm shown in FIG. 3 with the second flow diverter placed;

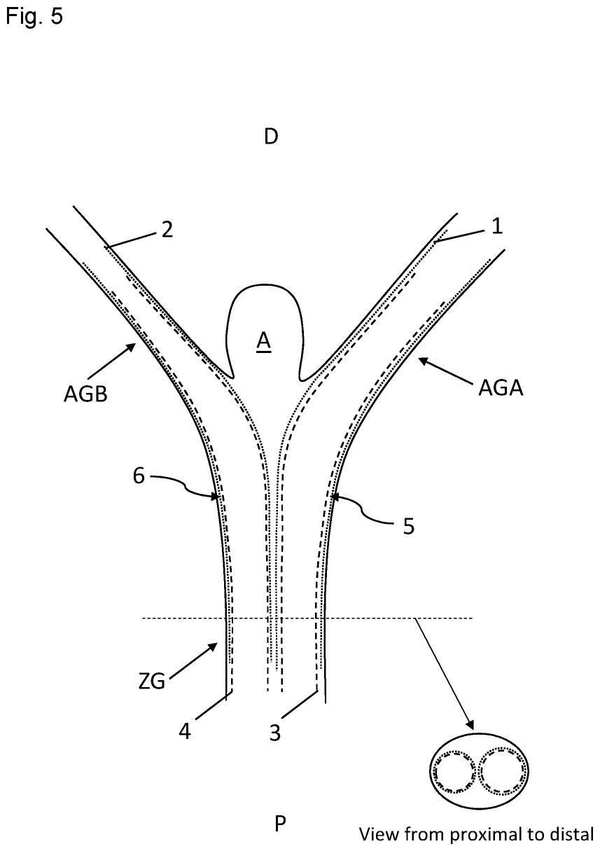

FIG. 5 the bifurcation aneurysm shown in FIG. 4 with the stents placed in the flow diverters;

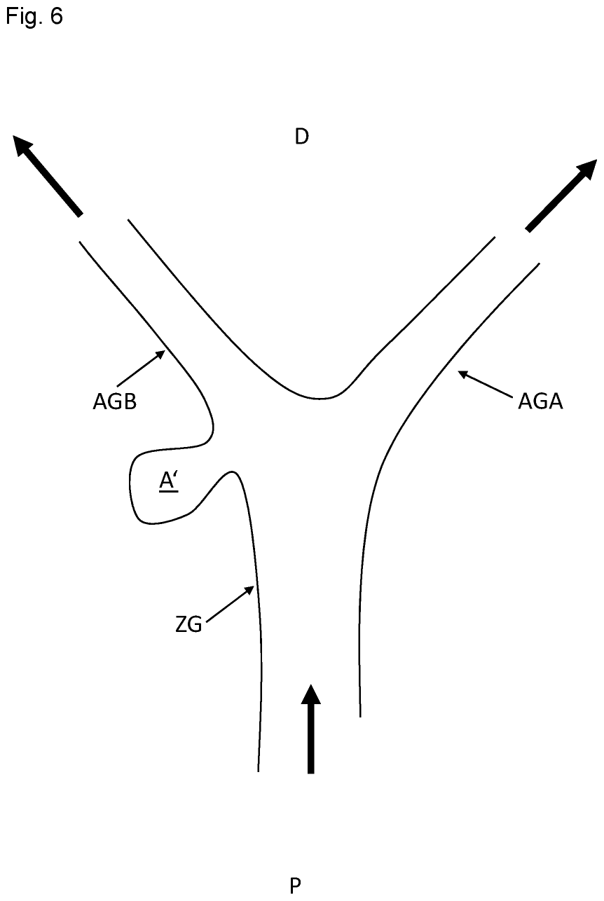

FIG. 6 an alternative position of the bifurcation aneurysm;

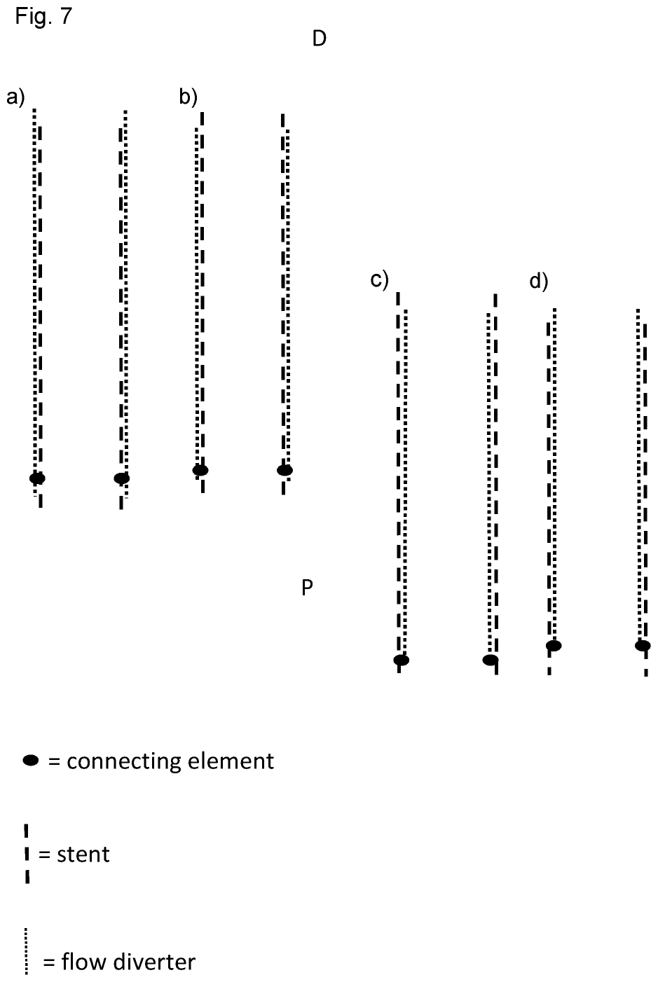

FIG. 7 a preferred embodiment of the connected combination implant with a stent.

FIG. 1 shows a schematic representation of a bifurcation with the inflowing vessel ZG and the two outflowing vessels AGA, AGB. The aneurysm A is located in the forking between the two outflowing vessels AGA, AGB.

FIG. 2 shows the bifurcation from FIG. 1, wherein two microcatheters MKA, MKB have been placed through the inflowing vessel ZG into the outflowing vessels AGA, AGB. One microcatheter MKA, MKB each is placed in each of the outflowing vessels AGA, AGB.

FIG. 3 shows how the first flow diverter 1 was placed, wherein the proximal end of the flow diverter 1 lies in the inflowing vessel ZG and the distal end of the flow diverter 1 lies in the outflowing vessel AGA. The diameter of the flow diverter 1 is selected so that it lies against the vessel wall of the outflowing vessel AGA. Accordingly, the diameter of the flow diverter 1 is smaller than the diameter of the inflowing vessel ZG, so that the flow diverter 1 can expand more strongly there. The microcatheters MKA, MKB remain in the vessels or the microcatheter MKA was reinserted again after placement of the first flow diverter 1.

FIG. 4 shows how the second flow diverter 2 was placed, wherein the proximal end of the flow diverter 2 is located in the inflowing vessel ZG and the distal end of the flow diverter 2 is located in the outflowing vessel AGB. The diameter of the flow diverter 2 is selected so that it lies against the vessel wall of the outflowing vessel AGB. Accordingly, the diameter of the flow diverter 2 is smaller than the diameter of the inflowing vessel ZG. Due to the dominant widening of the proximal section of the first flow diverter 1 in the inflowing vessel ZG, the second flow diverter 2 can no longer develop sufficiently there. Without additional measures, the outflowing vessel AGB would be increasingly undersupplied. The microcatheters MKA, MKB remain in the vessels or the microcatheter MKB was reinserted again after placement of the second flow diverter 2.

Alternatively, it is also conceivable that both flow diverters 1, 2 are implanted in parallel or simultaneously and not sequentially.

FIG. 5 shows how a stent 3, 4 was placed in each of the flow diverters 1, 2. The stents 3, 4 can be placed in parallel or simultaneously, but they can also be placed sequentially or one after the other. The proximal ends of the stents 3, 4 each lie in the inflowing vessel ZG and the distal ends of the stents 3, 4 each lie in the outflowing vessels AGA, AGB.

The first flow diverter 1 and the first stent 3 form a first functional unit, namely the first combination implant 5, and the second flow diverter 2 and the second stent 4 form a second functional unit, namely the second combination implant 6. It is essential for the invention of the implant system comprising the first combination implant 5 and the second combination implant 6 that both combination implants 5, 6 also form the same radial forces with the same deployment diameter. The radial forces of the stents 3, 4 should each advantageously be selected so large that the radial forces of the stents 3, 4 prevail over the radial forces of the flow diverters 1, 2 and the radial forces of the two combination implants 5, 6 are essentially determined by the radial forces of the stents 3, 4. This results in the two combination implants 5, 6 of the implant system permanently having a proximal deployment diameter of approximately the same size.

FIG. 6 shows an alternative position of an aneurysm A′ in a bifurcation, wherein the aneurysm A′ is not located between the outflowing vessels AGA, AGB, but between the inflowing vessel ZG and an outflowing vessel AGB. An aneurysm located in this way can also be treated analogously with the implant system according to the invention as shown in FIGS. 1 to 5.

FIGS. 7 a) to d) show variants of a preferred embodiment of a connected combination implant. In contrast to the unconnected combination implants 5, 6 shown in FIGS. 1 to 5, which essentially consist of separate flow diverters 1, 2 and stents 3, 4 and can therefore be implanted one after the other or in parallel and are physically present as individual implants, the connected combination implants shown below already represent physically connected combination implants outside the patient.

FIGS. 7 a) to d) show connected combination implants in which the flow diverters and stents are connected to each other in the proximal region.

The stent protrudes slightly beyond the flow diverter in the proximal area. This is advantageous in order to prevent a fish mouth effect, i.e. a proximal narrowing of the flow diverter. The stent can optionally also protrude beyond the flow diverter in the distal area b), c). It is important in this embodiment that the stent is provided at least over the section of the combination implant over which the two combination implants touch in a bifurcation during implantation. This is generally the area from the proximal beginning of the implant all the way into the outflowing vessel.

Preferably, the flow diverter is provided outside the stent a), b), but embodiments in which the stent is provided outside the flow diverter c), d) are also conceivable. In the second case, it is advantageous to provide the connection between the stent and the flow diverter in such a way that no fish-mouth effect can occur within the combination implant. The connection is then preferably located at the outermost proximal end of the flow diverter.

Variants of this embodiment are conceivable in which the flow diverter and stent are interwoven proximally, i.e. the connection is in a kind of entanglement.

### LIST OF REFERENCE SYMBOLS

1 first flow diverter
2 second flow diverter
3 first stent
4 second stent
5 first combination implant
6 second combination implant
ZG inflowing vessel
AG outflowing vessel (AGA: first outflowing vessel; AGB: second outflowing vessel)
A, A′ aneurysm (bifurcation aneurysm)
MK microcatheter (MKA: first microcatheter; MKB: second microcatheter)
D distal
P proximal

1. A combination implant comprising a flow diverter (1, 2) and at least one stent (3, 4), characterized in that the flow diverter (1, 2) and the stent (3, 4) form a functional unit (5, 6) and the radial force of the stent (3, 4) is greater than the radial force of the flow diverter (1, 2).

2. A combination implant according to claim 1, characterized in that the stent (3, 4) is provided within the lumen of the flow diverter (1, 2).

3. A combination implant according to claim 1, characterized in that the flow diverter (1, 2) is provided within the lumen of the stent (3, 4).

4. A combination implant according to claim 1, characterized in that the flow diverter (1, 2) is braided from 32 to 128 wires and preferably from 48 to 64 wires.

5. A combination implant according to claim 1, characterized in that the stent (3, 4) is provided to be self-expandable or balloon-expandable.

6. A combination implant according to claim 1, characterized in that the flow diverter (1, 2) and the at least one stent (3, 4) are connected proximally to each other.

7. A combination implant according to claim 6, characterized in that the flow diverter (1, 2) and the at least one stent (3, 4) are welded, glued, knotted, riveted or soldered together proximally.

8. An implant system for use in the treatment of aneurysms (A, A′) comprising at least two combination implants (5, 6) according to claim 1, characterized in that the radial force A of the first combination implant (5) and the radial force B of the second combination implant (6) are at least proximally equal with the same deployment diameter of the combination implants (5, 6).

9. A method for placing an implant system according to claim 8 comprising two combination implants (5, 6) in the treatment of a bifurcation aneurysm (A, A′) wherein each of the two combination implants comprise:
a flow diverter (1, 2) and at least one stent (3, 4), characterized in that the flow diverter (1, 2) and the stent (3, 4) form a functional unit (5, 6) and the radial force of the stent (3, 4) is greater than the radial force of the flow diverter (1, 2), wherein said stent (3, 4) is disposed within the lumen of the flow diverter (1, 2);
said method comprising the following steps:
(A) Placement of one microcatheter each (MKA, MKB) proximal through the vessel (ZG) inflowing in one outflowing vessel each (AGA, AGB);
(B) Sequential or parallel implantation of the flow diverter or the flow diverters (1, 2), wherein the proximal end of the respective flow diverter (1, 2) is located in the inflowing vessel (ZG) and the distal end of the respective flow diverter (1, 2) is located in the respective outflowing vessel (AGA, AGB);
(C) Sequential or parallel implantation of the stent or the stents (3, 4) in each case within the corresponding flow diverter (1, 2), wherein the proximal end of the respective stent (3, 4) lies proximal to the proximal end of the respective flow diverter (1, 2) and the distal end of the respective stent (3, 4) lies in the respective outflowing vessel (AGA, AGB).

10. A method of placing an implant system according to claim 8 comprising two combination implants (5, 6) in the treatment of a bifurcation aneurysm (A, A′) wherein each of the two combination implants comprise:
a flow diverter (1, 2) and at least one stent (3, 4), characterized in that the flow diverter (1, 2) and the stent (3, 4) form a functional unit (5, 6) and the radial force of the stent (3, 4) is greater than the radial force of the flow diverter (1, 2), wherein said flow diverter (1, 2) is disposed within the lumen of the stent (3, 4);
said method comprising the following steps:
(AA) Placement of one microcatheter each (MKA, MKB) from proximal through the inflowing vessel (ZG) in one outflowing vessel each (AGA, AGB);
(BB) Sequential or parallel implantation of the stent or stents (3, 4), wherein the proximal end of the respective stent (3, 4) lies in the inflowing vessel (ZG) and the distal end of the respective stent (3, 4) lies in the respective outflowing vessel;
(CC) Sequential or parallel implantation of the flow diverter or the flow diverters (1, 2) each within the corresponding stent (3, 4), wherein the proximal end of the respective flow diverter (1, 2) lies proximal to the proximal end of the respective stent (3, 4) and the distal end of the respective flow diverter (1, 2) lies in the respective outflowing vessel (AGA, AGB).

11. A method of placing an implant system according to claim 8 comprising two connected combination implants in the treatment of a bifurcation aneurysm (A, A′) wherein each of the two combination implants comprise:
a flow diverter (1, 2) and at least one stent (3, 4), characterized in that the flow diverter (1, 2) and the stent (3, 4) form a functional unit (5, 6) and the radial force of the stent (3, 4) is greater than the radial force of the flow diverter (1, 2), wherein said flow diverter (1, 2) is disposed within the lumen of the stent (3, 4);
said method comprising the following steps:
(AAA) Placement of one microcatheter each (MKA, MKB) from proximal through the inflowing vessel (ZG) in one outflowing vessel each (AGA, AGB);
(BBB) Sequential or parallel implantation of the connected combination implants, wherein the proximal end of the respective combination implant is located in the inflowing vessel and the distal end of the respective combination implant is located in the respective outflowing vessel.
